(19) United States
(12) Patent Application Publication
(10) Pub. No.: US 2021/0389293 A1
(43) Pub. Date: Dec. 16, 2021

(54) Methods and Systems for Water Area Pollution Intelligent Monitoring and Analysis

(71) Applicant: CHINESE RESEARCH ACADEMY OF ENVIRONMENTAL SCIENCES, Beijing (CN)

(72) Inventors: LIEYU ZHANG, Beijing (CN); RENHAO TANG, Beijing (CN); XIAOSHU WEI, Beijing (CN); XIAOGUANG LI, Beijing (CN); GUOWEN LI, Beijing (CN); CHEN ZHAO, Beijing (CN); JIAQIAN LI, Beijing (CN); WEI LI, BEIJING (CN)

(21) Appl. No.: 17/240,236

(22) Filed: Apr. 26, 2021

(30) Foreign Application Priority Data
Jun. 12, 2020 (CN) 2020105377654

(51) Int. Cl. G01N 33/18; G06K 9/46; G06T 7/00

(52) U.S. Cl. CPC G01N 33/1886; G06K 9/4619; G06T 2207/30181; G06T 2207/20084; G06T 7/0012

(57) ABSTRACT

Embodiments of the present disclosure relate to a water area pollution intelligent monitoring and analysis method and system. The method includes: acquiring, by a receiving module of a water area pollution monitoring and analysis device, water quality data of a monitored water area; inputting, by a processing module in the water area pollution monitoring and analysis device, the water quality data into a water quality feature extraction model in the processing module to obtain water quality features of the monitored water area, where the water quality feature extraction model is previously trained; determining, by an encoding module in the water area pollution monitoring and analysis device, a state diagram of the monitored water area according to the water quality features; and sending, by a routing module in the water area pollution monitoring and analysis device, the state diagram to a preset server.

### CROSS REFERENCE TO RELATED APPLICATION

This application claims the benefit of Chinese Patent Application No. 2020105377654, filed Jun. 12, 2020, which is hereby incorporated by reference herein in its entirety.

### TECHNICAL FIELD

The present disclosure relates to the field of environmental protection, in particular to a water area pollution intelligent monitoring and analysis method and system.

### BACKGROUND

Most of current water area pollution monitoring methods use sensors to collect data and then directly use statistical methods to analyze characteristics of a water area and average changes over a period of time.

As shown in FIG. 1, data is collected from a monitored water area and packaged into data packets, the data packets are transmitted through a network to a data center for processing, and finally streamlined results after analysis are returned to a terminal of a local environmental department to provide decision-making reference.

In the existing methods, due to weak data processing capability, a collection end is often unable to undertake complex analysis and computing tasks, so that the data needs to be uploaded to a remote computing center. Although such methods may improve the data analysis capability, they have the following defects:

Firstly, the data center is generally not located locally, packaging and network transmission of the data consumes lots of time, the transmission cost is high, and data analysis results lose their timeliness, thereby affecting the response speed of the local environmental department.

Secondly, the data collection end and a processing end are separated, and the dimensions of the data that can be transmitted are limited, so that the situation of the data collection end cannot be completely acquired by the processing end. Due to the loss of information, the existing methods affect the effect of data analysis.

Finally, there are security problems in transmission through a network. The data may be intercepted and tampered with by criminals, and the strong dependence on the network leads to increased difficulties in deployment.

For the above problems, no effective solutions have been proposed yet.

### SUMMARY

Embodiments of the present disclosure provide a water area pollution intelligent monitoring and analysis method and system, to at least solve the technical problems of low data transmission speed and processing speed and poor security due to the separation of the data collection end and the processing end in the water area pollution monitoring and analysis system.

According to one aspect of the embodiments of the present disclosure, there is provided a water area pollution intelligent monitoring and analysis method, applied to a water area pollution monitoring and analysis device system. The method includes: acquiring, by a receiving module of the water area pollution monitoring and analysis system device, a plurality of sets of water quality data of a monitored water area at a preset time interval; respectively inputting, by a processing module in the water area pollution monitoring and analysis system device, the plurality of sets of water quality data into a water quality feature extraction model in the processing module to obtain a plurality of sets of water quality features of the monitored water area, where the water quality feature extraction model is previously trained; determining, by an encoding module in the water area pollution monitoring and analysis system device, a state diagram of the monitored water area according to the plurality of sets of water quality features; and sending, by a routing module in the water area pollution monitoring and analysis system device, the state diagram to a preset server.

Further, the acquiring, by the receiving module of the water area pollution monitoring and analysis system device, the plurality of sets of water quality data of the monitored water area at the preset time interval includes: receiving and acquiring, by the receiving module, a water quality image of the monitored water area and sensor data collected by a sensor in the monitored water area at the preset time interval; and converting, by a processing unit, the water quality image and the sensor data into a preset format to obtain the water quality data.

Further, the sensor data includes at least one of the following: water temperature, water quality, flow velocity, animal and plant data in water, and pollution identification substance content in water.

Further, the water quality feature extraction model is composed of a convolutional neural network.

Further, the inputting, by the processing module in the water area pollution monitoring and analysis device, the water quality data into the water quality feature extraction model in the processing module includes converting, by the processing unit, the water quality image into the preset format to obtain water quality image data; and inputting the water quality image data into the convolutional neural network to obtain the water quality features.

Further, the determining, by the encoding module in the water area pollution monitoring and analysis system device, the state diagram of the monitored water area according to the plurality of sets of water quality features includes: superimposing and encoding, by the encoding module, the water quality features and geographic location information of the monitored water area to obtain a first state diagram of the monitored water area.

Further, the sending, by the routing module in the water area pollution monitoring and analysis system device, the state diagram to the preset server includes sending, by the routing module, the first state diagram to a cloud server.

Further, the determining, by the encoding module in the water area pollution monitoring and analysis system device, the state diagram of the monitored water area according to the plurality of sets of water quality features includes: classifying, by a cluster analysis module in the water area pollution monitoring and analysis system device, the water quality features and the sensor data; and superimposing, by the encoding module, the classified water quality features and the geographic system device information to obtain a second state diagram of the monitored water area, where the second state diagram includes classification information of the water quality features.

Further, the sending, by the routing module in the water area pollution monitoring and analysis system device, the state diagram to the preset server includes sending, by the routing module, the second state diagram and the corresponding sensor data to a local server and a cloud server.

Further, after the sending, by the routing module in the water area pollution monitoring and analysis system, the state diagram to the preset server, the method further includes maintaining, by the routing module, a routing table between the routing module and the preset server.

According to another aspect of the embodiments of the present disclosure, there is further provided a water area pollution intelligent monitoring and analysis system, including: a receiving module, a processing module, an encoding module and a routing module, where: the receiving module is configured to acquire a plurality of sets of water quality data of a monitored water area; the processing module is configured to respectively input the plurality of sets of water quality data into a water quality feature extraction model in the processing module to obtain a plurality of sets of water quality features of the monitored water area, where the water quality feature extraction model is previously trained; the encoding module is configured to determine a state diagram of the monitored water area according to the plurality of sets of water quality features; and the routing module is configured to send the state diagram to the preset server.

In the embodiments of the present disclosure, the plurality of sets of water quality data of the monitored water area are acquired by the receiving module of the water area pollution intelligent monitoring and analysis system; the plurality of sets of water quality data are respectively input by the processing module in the water area pollution monitoring and analysis system device into the water quality feature extraction model in the processing module to obtain the plurality of sets of water quality features of the monitored water area, where the water quality feature extraction model is previously trained; the state diagram of the monitored water area is determined by the encoding module in the water area pollution monitoring and analysis system device according to the plurality of sets of water quality features; and the state diagram is sent by the routing module in the water area pollution monitoring and analysis system device to the preset server. The objective of performing data processing at the data collection end is achieved, thereby realizing the technical effect of increasing the data transmission speed and processing speed, enhancing the security in the data transmission process, and further solving the technical problems of low data transmission speed and processing speed and poor security due to the separation of the data collection end and the processing end in the water area pollution monitoring and analysis system.

### BRIEF DESCRIPTION OF THE DRAWINGS

In order to more clearly illustrate the technical solutions of embodiments of the present disclosure, the accompanying drawings that need to be used in the description of the embodiments or the prior art will be briefly described below. Obviously, the accompanying drawings in the following description are only some embodiments of the present disclosure, and those of ordinary skill in the art can obtain other accompanying drawings according to these accompanying drawings without any creative effort.

FIG. 1 is a schematic diagram of a water area pollution intelligent monitoring and analysis process according to the prior art;

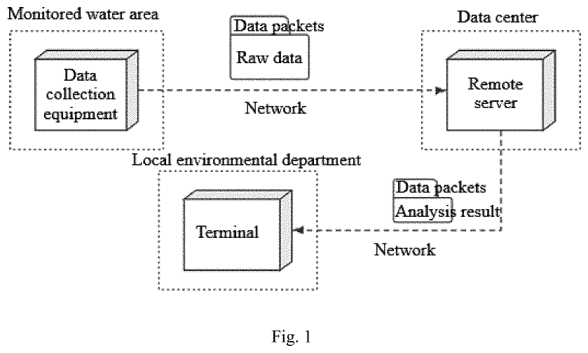

FIG. 2 is a schematic diagram of an optional water area pollution intelligent monitoring and analysis device according to an embodiment of the present disclosure;

FIG. 3 is a schematic diagram of an optional water area pollution intelligent monitoring and analysis method according to an embodiment of the present disclosure; and

FIG. 4 is a schematic diagram of an optional water area pollution intelligent monitoring and analysis system according to an embodiment of the present disclosure.

### DETAILED DESCRIPTION

In order to make the objectives, technical solutions and advantages of the embodiments of the present disclosure clearer, the technical solutions in the embodiments of the present disclosure will be clearly and completely described below in conjunction with the accompanying drawings in the embodiments of the present disclosure. It is apparent that the described embodiments are a part of the embodiments of the present disclosure, rather than all of the embodiments. All other embodiments obtained by those of ordinary skill in the art based on the embodiments in the present disclosure without creative efforts shall fall within the protection scope of the present disclosure.

It should be noted that relational terms such as “first” and “second” herein are only used to distinguish one entity or operation from another entity or operation, and do not necessarily require or imply any such actual relationship or sequence between these entities or operations.

### Embodiment 1

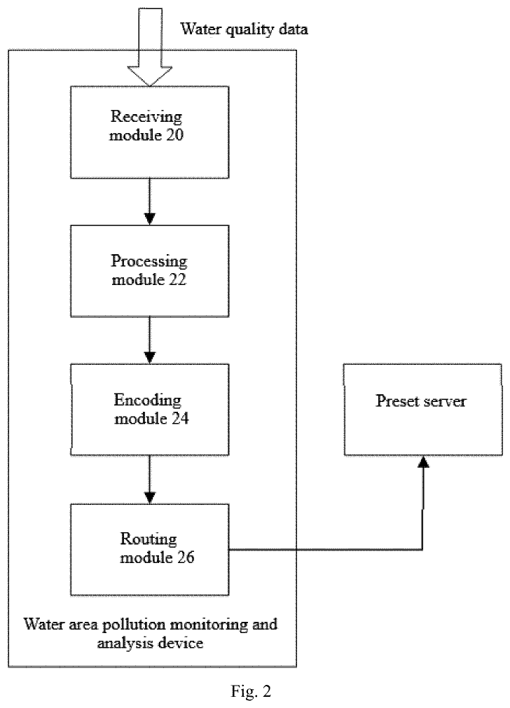

Before introducing a water area pollution intelligent monitoring and analysis method of this embodiment, a water area pollution intelligent monitoring and analysis system is firstly introduced. As shown in FIG. 2, the system includes: a receiving module 20, a processing module 22, an encoding module 24 and a routing module 26, where:

1) the receiving module 20 is configured to acquire water quality data of a monitored water area;

2) the processing module 22 is configured to input the water quality data into a water quality feature extraction model in the processing module 22 to obtain water quality features of the monitored water area, where the water quality feature extraction model is previously trained;

3) the encoding module 24 is configured to determine a state diagram of the monitored water area according to the water quality features; and

4) the routing module 26 is configured to send the state diagram to a preset server.

In a specific application scenario, the receiving module 20 is configured to receive sensor data collected by a sensor in the monitored water area and audio and video media data such as images and the like collected by an image collection device in the monitored water area.

In the process of the receiving module 20 receiving the sensor data and the audio media data sent by the sensor and the image collection device, used communication modes include, but are not limited to, Bluetooth, infrared, Zigbee, wireless network WIFI, near field communication NFC, wired transmission and the like, and communication networks between the routing module 26 and the preset server include, but are not limited to, a wide area network, a metropolitan area network, a local area network or the like.

The processing module 22 has certain storage resources and computing resources for storing data and executing computing tasks, so that the water quality data received by the processing module 22 can be processed.

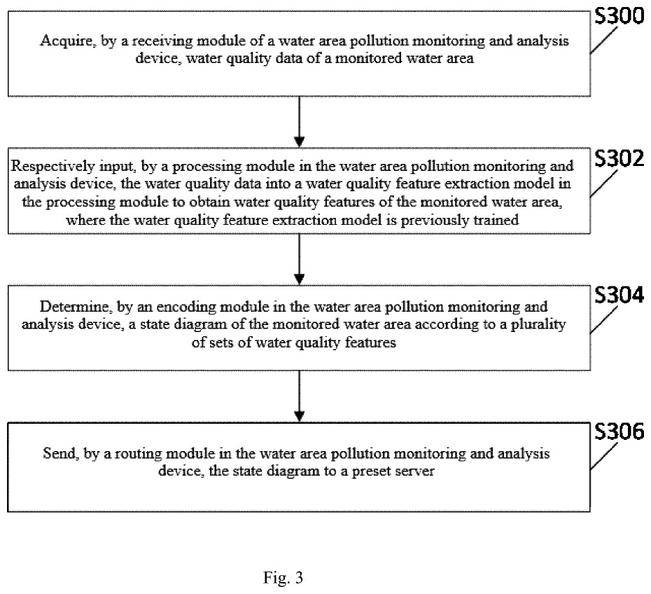

According to the embodiments of the present disclosure, there is provided a water area pollution monitoring and analysis method. As shown in FIG. 3, the method is applied to a water area pollution monitoring and analysis device and includes:

S300, a receiving module of the water area pollution monitoring and analysis device acquires water quality data of a monitored water area.

In a specific application scenario, in the monitored water area, the image collection device and the sensor in the monitored water area acquire data at the preset time interval. For example, the image collection device captures water surface images every 1 hour. For another example, the sensor collects the water quality data every 30 minutes. After the water quality data is collected, the water quality data can be transmitted in real time, and the water quality data may also be transmitted at another time interval, which is not limited here.

Optionally, in this embodiment, the acquiring, by the receiving module of the water area pollution monitoring and analysis device, the water quality data of the monitored water area includes, but is not limited to acquiring, by the receiving module, a water quality image of the monitored water area and sensor data collected by the sensor in the monitored water area at a preset time interval.

Specifically, the receiving module receives the water quality data sent by a data collection end at a preset time interval. For example, the water quality data sent by a sensor and a camera is acquired every 30 minutes. The water quality data includes a water quality image of the monitored water area and sensor data collected by the sensor.

Optionally, in this embodiment, the sensor data includes at least one of the following:

water temperature, water quality, flow velocity, animal and plant data in water, and pollution identification substance content in water.

In a specific application scenario, the sensor data includes water body color, water temperature, meteorological data, water quality, geographic location GIS information, flow velocity, animal and plant data in water, pollution identification substance content in water or the like. Specifically, the sensor data may also include code of the sensor, equipment status information of the sensor and the like. Water quality images collected by the image collection device include environmental images around the monitored water area, underwater images, microscopic images of water samples and the like, such as drain outlets of a factory, domestic sewage drainage pipes for residents, satellite images of the monitored water area and the like.

S302, a processing module in the water area pollution monitoring and analysis device respectively inputs the water quality data into the water quality feature extraction model in the processing module to obtain water quality features of the monitored water area, where the water quality feature extraction model is previously trained.

Optionally, in this embodiment, the water quality feature extraction model is composed of a convolutional neural network.

Optionally, in this embodiment, the inputting, by the processing module in the water area pollution monitoring and analysis device, the water quality data into the water quality feature extraction model in the processing module includes converting, by a processing unit, the water quality image into a preset format to obtain the water quality image data; and inputting the water quality image data into the convolutional neural network to obtain the water quality features.

In a specific application scenario, the water quality data received by the receiving module is generally binary data, or data in a data format previously negotiated with the data sensor and the image collection device, which is subjected to unified data format conversion in the processing module of the water area pollution monitoring and analysis device and converted into a unified data format that can be identified and processed by the processing module. Since the collected water quality data has time-domain characteristics, the image data is subjected to feature extraction through the convolutional neural network.

S304, an encoding module in the water area pollution monitoring and analysis device determines a state diagram of the monitored water area according to the plurality of sets of water quality features.

Optionally, in this embodiment, the determining, by the encoding module in the water area pollution monitoring and analysis device, the state diagram of the monitored water area according to the water quality features includes, but is not limited to the following two manners:

1) The water quality features and geographic location information of the monitored water area are superimposed and encoded by the encoding module to obtain a first state diagram of the monitored water area.

Specifically, after the water quality features of the water quality image are obtained from the convolutional neural network, the water quality features and the GIS information are superimposed and encoded to obtain the state diagram of the monitored water area, that is, water quality state of each location of the monitored water area is marked on the state diagram.

2) The water quality features and the sensor data are classified by a cluster analysis module in the water area pollution monitoring and analysis device; and the classified water quality features and the geographic location information are superimposed by the encoding module to obtain a second state diagram of the monitored water area, where the second state diagram includes classification information of the water quality features.

Specifically, after the water quality features of the water quality image are obtained from the convolutional neural network, the water quality features and the GIS information are superimposed, and then compared with the obtained sensor data to judge whether the predicted result of the sensor data is consistent with the predicted result of the water quality features. If so, it is considered that the sensor data is reliable, the sensor in the monitored water area is not under man-made interference, and there is no data falsification; and if not, it is considered that the sensor data is unreliable, the sensor is under man-made interference, and there is data falsification. The state diagrams and the sensor data are classified based on the judgment result. The second state diagrams without data falsification and the corresponding sensor data form one set, and the second state diagrams with data falsification and the corresponding sensor data form another set.

S306, a routing module in the water area pollution monitoring and analysis device sends the state diagram to a preset server.

Optionally, in this embodiment, the sending, by the routing module in the water area pollution monitoring and analysis device, the state diagram to the preset server includes, but is not limited to:

sending, by the routing module, the state diagram and the corresponding sensor data to a cloud server.

Specifically, the state diagram and the corresponding sensor data are sent to the cloud server for data storage so as to be processed by local personnel in time.

Optionally, in this embodiment, after the sending, by the routing module in the water area pollution monitoring and analysis device, the state diagram to the preset server, the method further includes, but is not limited to maintaining, by the routing module, a routing table between the routing module and the preset server.

In a specific application scenario, the cloud server also stores historical data reported by the routing module, that is historical records of the state diagram of the monitored area. In combination with real-time satellite remote sensing data, the GIS data in the state diagram of the routing module is used as a reference factor to identify, through a preset neural network algorithm, whether the judgment result of water quality in the state diagram is correct and whether there is data falsification. If there is data falsification, an alarm will be given in a preset manner to prompt that there is data falsification in the monitored water area corresponding to the state diagram.

It should be noted that according to the water area pollution monitoring and analysis method in this embodiment, the plurality of sets of water quality data of the monitored water area are acquired by the receiving module of the water area pollution intelligent monitoring and analysis device; the plurality of sets of water quality data are respectively input by the processing module in the water area pollution monitoring and analysis device into the water quality feature extraction model in the processing module to obtain the plurality of sets of water quality features of the monitored water area, where the water quality feature extraction model is previously trained; the state diagram of the monitored water area is determined by the encoding module in the water area pollution monitoring and analysis device according to the plurality of sets of water quality features; and the state diagram is sent by the routing module in the water area pollution monitoring and analysis device to the preset server. The objective of performing data processing at the data collection end is achieved, thereby realizing the technical effect of increasing the data transmission speed and processing speed, enhancing the security in the data transmission process, and further solving the technical problems of low data transmission speed and processing speed and poor security due to the separation of the data collection end and the processing end in the water area pollution monitoring and analysis device. It should be noted that for the foregoing method embodiments, for the sake of simple description, they are all expressed as a combination of a series of actions, but those skilled in the art should know that the present disclosure is not limited by the described sequence of actions, because according to the present disclosure, some steps can be performed in other sequence or simultaneously. Secondly, those skilled in the art should also know that the embodiments described in the specification are all preferred embodiments, and the actions and modules involved are not necessarily required by the present disclosure.

Through the description of the above implementations, those skilled in the art can clearly understand that the method according to the above embodiment can be implemented by means of software plus a necessary general hardware platform, and of course, it can also be implemented by hardware, but in many cases the former is a better implementation. Based on such an understanding, the technical solution of the present disclosure essentially or for the part that contributes to the prior art can be embodied in the form of a software product, and the computer software product is stored in a storage medium (such as ROM/RAM, a magnetic disk, an optical disk) and includes several instructions to enable terminal equipment (which may be a mobile phone, a computer, a server, network equipment or the like) to execute the method described in the embodiments of the present disclosure.

### Embodiment 2

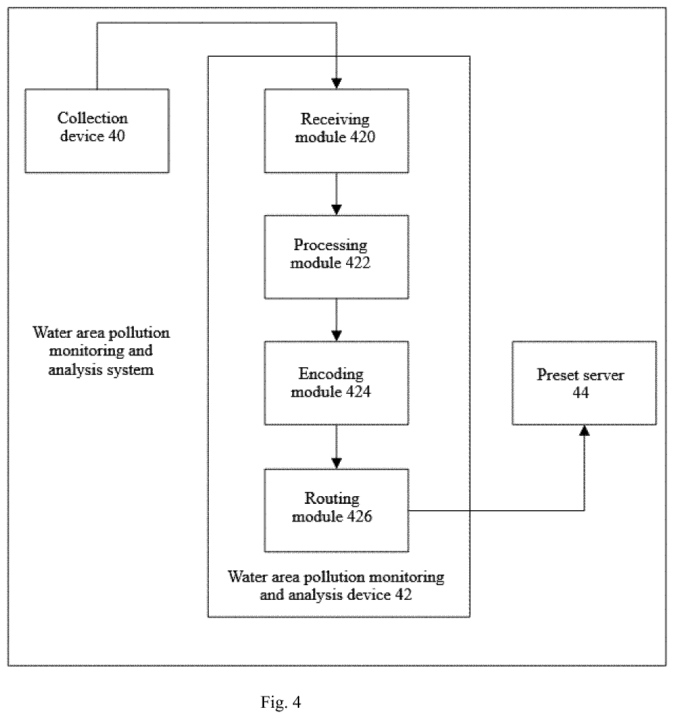

According to the embodiments of the present disclosure, there is further provided a water area pollution intelligent monitoring and analysis system. As shown in FIG. 4, the system includes: a water area pollution monitoring and analysis device 42, a collection device 40 and a preset server 44, where:

1) the collection device 40 is configured to collect water quality data of a monitored area;

2) the water area pollution monitoring and analysis device 42 includes: a receiving module 420, a processing module 422, an encoding module 424 and a routing module 426, where:

A) the receiving module 420 is configured to acquire a plurality of sets of water quality data of a monitored water area;

B) the processing module 422 is configured to respectively input the plurality of sets of water quality data into a water quality feature extraction model in the processing module 422 to obtain a plurality of sets of water quality features of the monitored water area, where the water quality feature extraction model is previously trained;

C) the encoding module 424 is configured to determine a state diagram of the monitored water area according to the plurality of sets of water quality features; and

D) the routing module 426 is configured to send the state diagram to the preset server 44.

When an integrated unit in the embodiment above is implemented in a form of a software function unit and sold or used as an independent product, the integrated unit may be stored in the computer-readable storage medium above. Based on such an understanding, the technical solution of the disclosure essentially or for the part that contributes to the prior art or all or part of the technical solution can be embodied in the form of a software product, and the computer software product is stored in a storage medium and includes several instructions configured to enable one or more pieces of computer equipment (which may be a personal computer, a server, network equipment or the like) to execute all or part of the steps of the methods of the embodiments of the present disclosure.

In the above embodiments of the present disclosure, the description for each embodiment has its own focus. For parts that are not described in detail in a certain embodiment, reference may be made to related descriptions of other embodiments.

In the several embodiments provided in the present application, it should be understood that a disclosed client can be implemented in other ways. The device embodiments described above are only schematic. For example, the division of units is only a division of logical functions. In an actual implementation, there may be other division manners. For example, a plurality of units or components may be combined or may be integrated into another system, or some features may be omitted or not executed. In addition, displayed or discussed mutual coupling or direct coupling or communication connection may be indirect coupling or communication connection through some interfaces, units or modules, and may be in electrical or other forms.

The units described as separate components may or may not be physically separated, and the components displayed as units may or may not be physical units, that is, they may be located in one place, or may be distributed in a plurality of network units. Part or all of the units may be selected according to actual needs to achieve the purposes of the solutions of the embodiments.

In addition, the function units in each embodiment of the present disclosure may be integrated into one processing unit, or each unit may exist alone physically, or two or more units may be integrated into one unit. The above integrated unit may be implemented in the form of hardware or implemented in the form of a software function unit.

The above description is only preferred implementations of the present disclosure. It should be noted that those of ordinary skill in the art may also make several improvements and modifications without departing from the principles of the present disclosure, and such improvements and modifications should also be regarded as the protection scope of the present disclosure.

1. A water area pollution intelligent monitoring and analysis method, applied to a water area pollution monitoring and analysis device, comprising:
acquiring, by a receiving module of the water area pollution monitoring and analysis device, water quality data of a monitored water area;
inputting, by a processing module in the water area pollution monitoring and analysis device, the water quality data into a water quality feature extraction model in the processing module to obtain water quality features of the monitored water area, wherein the water quality feature extraction model is previously trained;
determining, by an encoding module in the water area pollution monitoring and analysis device, a state diagram of the monitored water area according to the water quality features; and
sending, by a routing module in the water area pollution monitoring and analysis device, the state diagram to a preset server.

2. The method according to claim 1, wherein the acquiring, by the receiving module of the water area pollution monitoring and analysis device, the water quality data of the monitored water area comprises:
acquiring, by the receiving module, a water quality image of the monitored water area and sensor data collected by a sensor in the monitored water area.

3. The method according to claim 2, wherein the sensor data comprises at least one of the following:
water temperature, water quality, flow velocity, animal and plant data in water, and pollution identification substance content in water.

4. The method according to claim 1, wherein the water quality feature extraction model is a convolutional neural network.

5. The method according to claim 4, wherein the inputting, by the processing module in the water area pollution monitoring and analysis device, the water quality data into the water quality feature extraction model in the processing module comprises:
converting, by a processing unit, the water quality image into a preset format to obtain water quality image data; and
inputting the water quality image data into the convolutional neural network to obtain the water quality features.

6. The method according to claim 2, wherein the determining, by the encoding module in the water area pollution monitoring and analysis device, the state diagram of the monitored water area according to the water quality features comprises:
superimposing and encoding, by the encoding module, the water quality features and geographic location information of the monitored water area to obtain a first state diagram of the monitored water area.

7. The method according to claim 6, wherein the determining, by the encoding module in the water area pollution monitoring and analysis device, the state diagram of the monitored water area according to the water quality features comprises:
classifying, by a cluster analysis module in the water area pollution monitoring and analysis device, the water quality features and the sensor data; and
superimposing, by the encoding module, the classified water quality features and the geographic location information to obtain a second state diagram of the monitored water area,
wherein the second state diagram comprises classification information of the water quality features.

8. The method according to claim 2, wherein the sending, by the routing module in the water area pollution monitoring and analysis device, the state diagram to the preset server comprises:
sending, by the routing module, the state diagram and the corresponding sensor data to a local server and a cloud server.

9. The method according to claim 8, wherein after the sending, by the routing module in the water area pollution monitoring and analysis device, the state diagram to the preset server, the method further comprises:
maintaining, by the routing module, a routing table between the routing module and the preset server.

10. A water area pollution intelligent monitoring and analysis system, comprising a water area pollution monitoring and analysis device, a collection device and a preset server, wherein:
the collection device is configured to collect water quality data of a monitored area;
the water area pollution monitoring and analysis device comprises: a receiving module, a processing module, an encoding module and a routing module, wherein:
the receiving module is configured to acquire the water quality data of the monitored water area;
the processing module is configured to input the water quality data into a water quality feature extraction model in the processing module to obtain water quality features of the monitored water area, wherein the water quality feature extraction model is previously trained;
the encoding module is configured to determine a state diagram of the monitored water area according to the water quality features; and
the routing module is configured to send the state diagram to the preset server.
